SEQUENCE LISTING (21 sequences)

SEQ ID NO: 1 - DNA, 1443 bp, Pyrococcus furiosus
atgggaatgagagttttggtaactacatggggtaatcccttccagtgggaaccaataacatatgaatacagaggaatcaaagttaaaagcagaaataccttgccaattctagtcaagactcttgagccagagaggattctaatccttgtggctgatacaatggccaactactatgattcaggaaaaaataagccagaaatagaagaaaaatcgttttcgtcttattcggaagttgtggaagatacaaaagaaaggatactatggcacataaaagaggaggtcattgaagaactccgtgaggaagatcctgagcttgctaagaaaattgagaatatgttaaaagatgaaagaattacaattgaagttcttcccggcgttggagtctttggcaacattacagtagagggagaaatgcttgacttctattattatgccacatacaagttggccgaatggttgccagttcagaacaatttagaggtttacttagacctaactcatgggataaatttcatgcccacctttacttacagagccctaagaaacttgcttggattgttggcctacttgtacaatgtaaagtttgagatagttaattcagaaccttatcccctgggggtttcacaagaaataagggaggacacaattctccatattagggaaattggagagggagtagttcgtcctagaccacagtattctccagtagaaggaaagctttactggaatgcatttataagctctgtagccaatggcttcccgttagtctttgccagcttttatccaaatattcgggacgtagaagattaccttaacaaaaagcttgaggaattcctggtgggaattgaggttggggagagagaagatggaaaaccttatgttaaaagagagaaagctcttgacaggagctttaagaatgcttctaagctctactatgctttaagagtgttcaatacaaaattccaaaactatccaaaaaaagaagttcctattgaagaaataatggagatatcaaagatattcgagtctcttcccaggattggaattattttagagaggcaagtagagtggctaagaaatttagtatatggaagattatggtatgaaaatggagaacagaaaataaagaagggtcttttagagattatcaaggataagaaggataaaaggaaagaggccgaagctcttaaaaaagggaagacaatatctttagccgaagctgcaaagcttacaagaatattttctccgagtggagaaagaatagagacaatagaatctccaaatgttgttcgtaactttatagcacattctggatttgagtataacattgtctatgtgaaatatgatagactaagtgataggctgtactttttctataaggataaagaaaaagctgcaaatctcgcttatgaagcccttttatataggggtgaaaaagaatga

SEQ ID NO: 2 - protein, 480 aa, Pyrococcus furiosus
MGMRVLVTTWGNPFQWEPITYEYRGIKVKSRNTLPILVKTLEPERILILVADTMANYYDSGKNKPEIEEKSFSSYSEVVEDTKERILWHIKEEVIEELREEDPELAKKIENMLKDERITIEVLPGVGVFGNITVEGEMLDFYYYATYKLAEWLPVQNNLEVYLDLTHGINFMPTFTYRALRNLLGLLAYLYNVKFEIVNSEPYPLGVSQEIREDTILHIREIGEGVVRPRPQYSPVEGKLYWNAFISSVANGFPLVFASFYPNIRDVEDYLNKKLEEFLVGIEVGEREDGKPYVKREKALDRSFKNASKLYYALRVFNTKFQNYPKKEVPIEEIMEISKIFESLPRIGIILERQVEWLRNLVYGRLWYENGEQKIKKGLLEIIKDKKDKRKEAEALKKGKTISLAEAAKLTRIFSPSGERIETIESPNVVRNFIAHSGFEYNIVYVKYDRLSDRLYFFYKDKEKAANLAYEALLYRGEKE

SEQ ID NO: 3 - RNA, 37 nt, Artificial
  Primer
cugaagugcucucagccgcaaggaccgcauacuacaa

SEQ ID NO: 4 - RNA, 37 nt, Artificial
  Primer
uuguaguaugcgguccuugcggcugagagcacuucag

SEQ ID NO: 5 - RNA, 45 nt, Artificial
  Primer
auugaaaguuguaguaugcgguccuugcggcugagagcacuucag

SEQ ID NO: 6 - RNA, 45 nt, Artificial
  Primer
auugaaagagggaauaagggcgacacggaaauguugaauacucau

SEQ ID NO: 7 - RNA, 45 nt, Artificial
  Primer
auugaaagagugaagaauuugacguacaaauguccuuaguggaac

SEQ ID NO: 8 - RNA, 67 nt, Artificial
  Primer
auugaaaguuguaguaugcgguccuugcggcugagagcacuucagucguuaucucuuacgaagucuu

SEQ ID NO: 9 - RNA, 19 nt, Artificial
  Primer
aaaaaaaaaaaaaaaaaaa

SEQ ID NO: 10 - RNA, 19 nt, Artificial
  Primer
ggggggggggggggggggg

SEQ ID NO: 11 - RNA, 19 nt, Artificial
  Primer
uuuuuuuuuuuuuuuuuuu

SEQ ID NO: 12 - RNA, 19 nt, Artificial
  Primer
ccccccccccaugaugaug

SEQ ID NO: 13 - DNA, 63 bp, Artificial
  Primer
atttaggtgacactatagattgaaagttgtagtatgcggtccttgcggctgagagcacttcag

SEQ ID NO: 14 - DNA, 63 bp, Artificial
  Primer
ctgaagtgctctcagccgcaaggaccgcatactacaactttcaatctatagtgtcacctaaat

SEQ ID NO: 15 - DNA, 45 bp, Artificial
  Primer
ctgaagtgctctcagccgcaaggaccgcatactacaactttcaat

SEQ ID NO: 16 - DNA, 52 bp, Artificial
  Primer
gacaatagaatctccaaatgttgttgctaactttatagcacattctggattt

SEQ ID NO: 17 - DNA, 52 bp, Artificial
  Primer
aaatccagaatgtgctataaagttagcaacaacatttggagattctattgtc

SEQ ID NO: 18 - DNA, 54 bp, Artificial
  Primer
caaatgttgttcgtaactttatagcagcttctggatttgagtataacattgtct

SEQ ID NO: 19 - DNA, 54 bp, Artificial
  Primer
agacaatgttatactcaaatccagaagctgctataaagttacgaacaacatttg

SEQ ID NO: 20 - DNA, 52 bp, Artificial
  Primer
gacaatagaatctccaaatgttgttgctaactttatagcagcttctggattt

SEQ ID NO: 21 - DNA, 52 bp, Artificial
  Primer
aaatccagaagctgctataaagttagcaacaacatttggagattctattgtc

(19) United States
(12) Patent Application Publication
(10) Pub. No.: US 2017/0191047 A1
(43) Pub. Date: Jul. 6, 2017

(54) ADENOSINE-SPECIFIC RNASE AND METHODS OF USE

(71) Applicant: UNIVERSITY OF GEORGIA RESEARCH FOUNDATION, INC., Athens, GA (US)

(72) Inventors: Rebecca M. Terns, Athens, GA (US); Michael P. Terns, Athens, GA (US); Nolan F. Sheppard, West Orange, NJ (US)

(21) Appl. No.: 15/353,559

(22) Filed: Nov. 16, 2016

Related U.S. Application Data
(60) Provisional application No. 62/255,164, filed on Nov. 13, 2015.

(51) Int. Cl. C12N 9/22; C12Q 1/68

(52) U.S. Cl. CPC C12N 9/22; C12Q 1/6876

(57) ABSTRACT

Provided herein are proteins having A-specific RNase activity. A protein having A-specific RNAse activity is referred to herein as a Csx1 protein. A Csx1 protein is an endoribonuclease, and has the activity of cleaving the phosphodiester bond in a single strand of a target RNA molecule on the 3′ (downstream) side of an adenosine base to result in a first cleavage product having a 5′ hydroxyl group and a second cleavage product having a 2′,3′-cyclic phosphate at the 3′ end. Also provided herein are methods for using a Csx1 protein. In one embodiment, the method includes incubating a sample that includes an isolated Csx1 protein and a target RNA molecule under suitable conditions for cleavage of the target RNA molecule. Also provided is a genetically modified microbe that includes an exogenous polynucleotide including a nucleotide sequence encoding a Csx1 protein, and a method for making Cxsl protein.

### CROSS-REFERENCE TO RELATED APPLICATIONS

This application claims the benefit of U.S. Provisional Application Ser. No. 62/255,164, filed Nov. 13, 2015, which is incorporated by reference herein.

### GOVERNMENT FUNDING

This invention was made with government support under GM54682, awarded by the National Institutes of Health. The government has certain rights in the invention.

### SEQUENCE LISTING

This application contains a Sequence Listing electronically submitted via EFS-Web to the United States Patent and Trademark Office as an ASCII text file entitled “235-02610101_SequenceListing_ST25.txt” having a size of 10 kilobytes and created on Nov. 10, 2016. The information contained in the Sequence Listing is incorporated by reference herein.

### SUMMARY OF THE APPLICATION

Provided herein are methods. In one embodiment, the method includes incubating a sample that includes an isolated Csx1 protein and a target RNA molecule including a single stranded region under suitable conditions for cleavage of the target RNA molecule by the Csx1 protein. Cleavage of the target RNA molecule occurs on the 3′ side of at least one adenosine residue of the target RNA molecule, and the cleavage. The target RNA molecule can be a single stranded RNA molecule, and the target RNA molecule can be linear.

In one embodiment, the target RNA molecule is from a biological sample, such as a microbial cell or a eukaryotic cell. In one embodiment, the target RNA molecule includes a label.

The method can further include detecting the presence or absence of cleavage of the target RNA molecule. In one embodiment, the method further including resolving the sample after the incubation under conditions suitable to separate from the target RNA molecule the at least one cleaved RNA molecule including an adenosine at the 3′ terminal end. In one embodiment, the conditions include denaturing polyacrylamide gel electrophoresis. In one embodiment, the method further including isolating the at least one cleaved RNA molecule including an adenosine at the 3′ terminal end.

In one embodiment, the method includes incubating a genetically modified cell that includes an exogenous polynucleotide including a nucleotide sequence encoding a protein having A-specific RNAse activity. In one embodiment, the amino acid sequence of the protein and the amino acid sequence of SEQ ID NO:2 have at least 85% identity. The incubation is under conditions suitable for expression of the protein. The method can further include isolating the protein.

In one embodiment, the genetically modified cell is a bacterium, such as E. coli, or an archaeon, such as a member of the genus Pyrococcus, for instance, P. furiosus.

Also provided is a genetically modified microbe including an exogenous protein. In one embodiment, the exogenous protein includes an amino acid sequence, wherein the amino acid sequence and the amino acid sequence of SEQ ID NO:2 have at least 85% identity. In one embodiment, the exogenous protein includes a heterologous amino acid sequence, such as a tag.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, enzyme, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, an “isolated” substance is one that has been removed from its natural environment, produced using recombinant techniques, or chemically or enzymatically synthesized. For instance, a protein or a polynucleotide can be isolated. Preferably, a substance is purified, i.e., is at least 60% free, preferably at least 75% free, and most preferably at least 90% free from other components with which they are naturally associated.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

As used herein, a “detectable moiety” or “label” is a molecule that is detectable, either directly or indirectly, by spectroscopic, photochemical, biochemical, immunochemical, or chemical means. For example, useful labels include ³²P, fluorescent dyes, electron-dense reagents, enzymes and their substrates (e.g., as commonly used in enzyme-linked immunoassays, e.g., alkaline phosphatase and horse radish peroxidase), biotin-streptavidin, digoxigenin, proteins such as antibodies, or haptens and proteins for which antisera or monoclonal antibodies are available. The label or detectable moiety is typically bound, either covalently, through a linker or chemical bound, or through ionic, van der Waals or hydrogen bonds to the molecule to be detected.

As used herein, the terms “coding region” and “coding sequence” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. Thus, a polynucleotide that includes a coding region and heterologous nucleotides is not a naturally occurring molecule. For instance, a coding region present in a wild-type microbe and encoding a Csx1 protein is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide described herein through the use of standard genetic and/or recombinant methodologies well known to one skilled in the art. A polynucleotide described herein may be included in a suitable vector.

A protein described herein may include heterologous amino acids present at the N-terminus, the C-terminus, or a combination thereof. As used herein, “heterologous amino acids” refer to amino acids that are not normally present flanking a protein that is naturally present in a wild-type cell. Thus, a protein that includes heterologous amino acids is not a naturally occurring molecule. For instance, a naturally occurring Csx1 protein present in a wild-type microbe does not have additional amino acids at either the N-terminal end or the C-terminal end, and any other amino acids present at the N-terminal end or the C-terminal end are considered to be heterologous. Examples of heterologous amino acid sequences are described herein, and include, but are not limited to affinity purification tags. Typically, heterologous amino acids are present in a protein described herein through the use of standard genetic and/or recombinant methodologies well known to one skilled in the art.

As used herein, an “exogenous polynucleotide” refers to a polynucleotide that is not normally or naturally found in a cell. As used herein, the term “endogenous polynucleotide” refers to a polynucleotide that is normally or naturally found in a cell. An “endogenous polynucleotide” is also referred to as a “native polynucleotide.”

The terms “complement” and “complementary” as used herein, refer to the ability of two single stranded polynucleotides to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The term “substantial complement” and cognates thereof as used herein refer to a polynucleotide that is capable of selectively hybridizing to a specified polynucleotide under stringent hybridization conditions. Stringent hybridization can take place under a number of pH, salt, and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically substantially complementary to a second polynucleotide if hybridization occurs between the polynucleotide and the second polynucleotide. As used herein, “specific hybridization” refers to hybridization between two polynucleotides under stringent hybridization conditions.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. The sequence similarity between two proteins is determined by aligning the residues of the two proteins (e.g., a candidate amino acid sequence and a reference amino acid sequence, such as SEQ ID NO:2) to optimize the number of identical amino acids along the lengths of their sequences; gaps in either or both sequences are permitted in making the alignment in order to optimize the number of shared amino acids, although the amino acids in each sequence must nonetheless remain in their proper order. Sequence similarity may be determined, for example, using sequence techniques such as the BESTFIT algorithm in the GCG package (Madison Wis.), or the Blastp program of the BLAST 2 search algorithm, as described by Tatusova, et al. (FEMS Microbiol Lett 1999, 174:247-250), and available through the World Wide Web, for instance at the interne site maintained by the National Center for Biotechnology Information, National Institutes of Health. Preferably, sequence similarity between two amino acid sequences is determined using the Blastp program of the BLAST 2 search algorithm. Preferably, the default values for all BLAST 2 search parameters are used. In the comparison of two amino acid sequences using the BLAST search algorithm, structural similarity is referred to as “identities.” Thus, reference to a protein described herein, such as SEQ ID NO:2, can include a protein with at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with the reference protein. Alternatively, reference to a protein described herein, such as SEQ ID NO:2, can include a protein with at least 80% similarity, at least 81% similarity, at least 82% similarity, at least 83% similarity, at least 84% similarity, at least 85% similarity, at least 86% similarity, at least 87% similarity, at least 88% similarity, at least 89% similarity, at least 90% similarity, at least 91% similarity, at least 92% similarity, at least 93% similarity, at least 94% similarity, at least 95% similarity, at least 96% similarity, at least 97% similarity, at least 98% similarity, or at least 99% similarity with the reference protein.

The sequence similarity between two polynucleotides is determined by aligning the residues of the two polynucleotides (e.g., a candidate nucleotide sequence and a reference nucleotide sequence, such as SEQ ID NO:1) to optimize the number of identical nucleotides along the lengths of their sequences; gaps in either or both sequences are permitted in making the alignment in order to optimize the number of shared nucleotides, although the nucleotides in each sequence must nonetheless remain in their proper order. Sequence similarity may be determined, for example, using sequence techniques such as GCG FastA (Genetics Computer Group, Madison, Wisconsin), MacVector 4.5 (Kodak/IBI software package) or other suitable sequencing programs or methods known in the art. Preferably, sequence similarity between two nucleotide sequences is determined using the Blastn program of the BLAST 2 search algorithm, as described by Tatusova, et al. (1999, FEMS Microbiol Lett., 174:247-250), and available through the World Wide Web, for instance at the internet site maintained by the National Center for Biotechnology Information, National Institutes of Health. Preferably, the default values for all BLAST 2 search parameters are used. In the comparison of two nucleotide sequences using the BLAST search algorithm, sequence similarity is referred to as “identities.” The sequence similarity is typically at least 50% identity, at least 55% identity, at least 60% identity, at least 65% identity, at least 70% identity, at least 75% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity.

Conditions that “allow” an event to occur or conditions that are “suitable” for an event to occur, such as an enzymatic reaction, or “suitable” conditions are conditions that do not prevent such events from occurring. Thus, these conditions permit, enhance, facilitate, and/or are conducive to the event. Such conditions, known in the art and described herein, may depend upon, for example, the enzyme being used.

As used herein, a protein “fragment” includes any protein which retains at least some of the activity of the corresponding native protein. Examples of fragments of proteins described herein include, but are not limited to, proteolytic fragments and deletion fragments.

As used herein, a “microbe” is a single celled organism that is a member of the domain Archaea or a member of the domain Bacteria.

As used herein, “genetically modified cell” refers to a cell into which has been introduced an exogenous polynucleotide, such as an expression vector. For example, a cell is a genetically modified cell by virtue of introduction into a suitable cell of an exogenous polynucleotide that is foreign to the cell. “Genetically modified cell” also refers to a cell that has been genetically manipulated such that endogenous nucleotides have been altered. For example, a cell is a genetically modified cell by virtue of introduction into a suitable cell of an alteration of endogenous nucleotides. An example of a genetically modified cell is one having an altered regulatory sequence, such as a promoter, to result in increased or decreased expression of an operably linked endogenous coding region.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

In the description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The description is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows a ribbon diagram of the Pfu Csx1 monomer (PDB 4EOG). (FIG. 1A) The N-terminal modified Rossmannoid fold/CARF domain, the C-terminal winged-helix-like domain/HEPN domain, and the highly conserved HEPN RxxxxH motif with predicted catalytic residues highlighted in black are shown. The dashed line represents 17 residues with missing electron density. (FIG. 1B) Isolated HEPN RxxxxH motif with predicted catalytic residues annotated.

FIG. 2 shows Csx1 is a temperature-dependent, single-strand-specific ribonuclease. (FIG. 2A) Csx1 was tested for nuclease activity (+) on −³²P-labeled single-stranded and double-stranded RNA and DNA (37mer A, 63mer A, 37mer A+B, and 63mer A+B, respectively), as well as RNA/DNA hybrids (45mer A+D), which were resolved by denaturing gel electrophoresis alongside no-protein controls (−). See Table 1 for RNA and DNA sequences. Asterisk indicates the labeled oligonucleotide. Size standard (M) is measured in nucleotides. Two lanes not contiguous in the original gel are juxtaposed (dotted lines). (FIG. 2B) −³²P-labeled ssRNA was incubated without (−) or with Csx1 across a range of temperatures, then resolved by denaturing gel electrophoresis. The arrow indicates the full-length RNA, while the bracket indicates Csx1 cleavage products.

FIG. 3 shows mutations of highly conserved residues in the HEPN domain affect RNase activity. (FIG. 3A) Radiolabeled ssRNA (37mer A) was incubated with no protein for 30 min (−), with wild-type (wt) or mutant Csx1 for 1 min (1) or for 30 min (30), followed by separation by denaturing gel electrophoresis. The arrow indicates the full-length RNA, while the bracket indicates Csx1 cleavage products. (FIG. 3B) Purified wt and mutant Csx1 proteins were analyzed by SDS-PAGE and Coomassie blue staining. Molecular weight marker is indicated in kilodaltons. (FIG. 3C) Csx1 cleavage activity occurs in the absence of added metal ions (−EDTA) and in the presence of a wide range (0.5, 1, 200, 500, 1000 μM) of EDTA. The dotted line separates data that was subject to longer exposure times to visualize molecular weight markers.

FIG. 4 shows endonucleolytic cleavage of ssRNA by Csx1. (FIG. 4A) Radiolabeled linear (L) and circular (C) ssRNAs (67mer) were incubated with no protein (−), Terminator 5′-phosphate-dependent exonuclease (TEX), or Csx1 for the indicated time, then resolved by denaturing gel electrophoresis. The full-length linear and circular RNA are indicated by arrows, while the Csx1 cleavage products are indicated by the bracket. (FIG. 4B) 5′-Radiolabeled RNA (45mer A) was treated with no protein (−), Csx1, poly(A) polymerase (PAP), or Csx1 followed by PAP, and resolved by denaturing gel electrophoresis. The arrow indicates RNA elongated by PAP, while the bracket indicates Csx1 cleavage products. (FIG. 4C) 3′-Radiolabeled RNA (45mer A) was treated with no protein (−), with Csx1, with TEX, or with Csx1 followed by Tex, while 5′-radiolabeled RNA was treated with or without TEX. The samples were resolved by denaturing gel electrophoresis. The arrow indicates the expected TEX cleavage product, while the bracket indicates Csx1 cleavage products. (FIG. 4D) A diagram depicting the cleavage method of RNA by Csx1 as suggested by the resistance of the cleavage products to TEX activity and protection from elongation by PAP.

FIG. 5 shows cleavage of homoribopolymers by Csx1. Radiolabeled RNA homoribopolymers of each ribonucleotide and an RNA composed of 10 cytidylate residues and three repeats of AUG were incubated with no protein (−) or Csx1 for the indicated times, then resolved by denaturing gel electrophoresis.

FIG. 6 shows Csx1 cleaves ssRNA after adenosines. (FIG. 6A) A variety of ssRNAs were treated with no protein (−) or Csx1 for the indicated times, and run alongside 5′-radiolabeled RNA markers (M), RNase T1 ladders (T1), and alkaline hydrolysis ladders (OH). The RNAs were resolved by denaturing sequencing gel electrophoresis. Arrows indicate Csx1 cleavage products. (FIG. 6B) Cleavage products were mapped back to their respective RNAs. Sites of cleavage are denoted with an A followed by a dash. No cleavage is mapped after the first A of 45mer B and C because the single nucleotide band was run off the gel. Comparison of the Csx1 ladders with the corresponding T1 ladders confirms that Csx1 cleavage occurs on the 3′ rather than 5′ side of adenosine. Poly(C₁₀) (AUG)₃, SEQ ID NO:12; 37mer A, SEQ ID NO:3; 45mer A, SEQ II) NO:5; 45mer B, SEQ ID NO:6; and 45mer C, SEQ II) NO:7.

FIG. 7 shows the amino acid sequence (SEQ ID NO:2) and an example of a nucleotide sequence (SEQ ID NO:1) encoding the amino acid sequence.

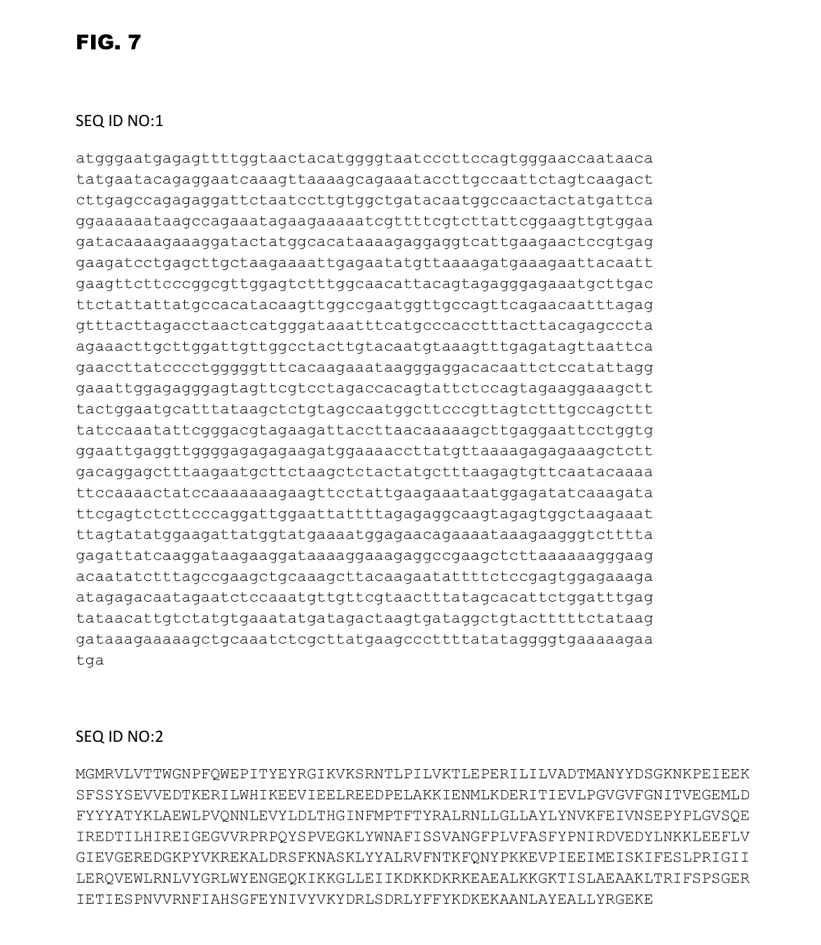

### DETAILED DESCRIPTION OF ILLUSTRATIVE EMBODIMENTS

The present invention includes isolated proteins having A-specific RNase activity. A protein having A-specific RNAse activity is referred to herein as a Csx1 protein. A protein having A-specific RNase activity catalytically cleaves under suitable conditions a target RNA molecule in a region that is single stranded. Thus, a target RNA molecule cleaved by a protein having A-specific RNase activity can be, for example, single stranded or double stranded with a region of strand separation. A target RNA molecule may be circular or linear. In one embodiment, a target RNA molecule may be part of a double stranded DNA-RNA hybrid. A single stranded RNA having one or more regions of secondary structure is considered a single stranded RNA. A target RNA may include a detectable moiety.

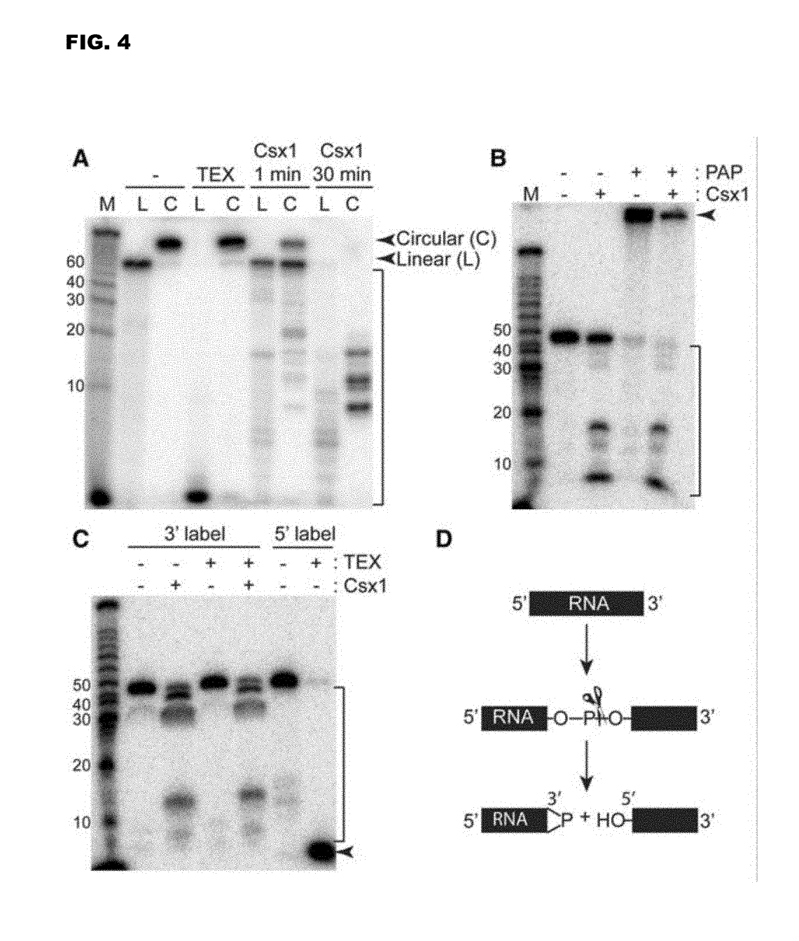

A Csx1 protein is an endoribonuclease, and has the activity of cleaving the phosphodiester bond in a single strand of a target RNA molecule on the 3′ (downstream) side of an adenosine base to result in a first cleavage product having a 5′ hydroxyl (OH) group and a second cleavage product having a 2′,3′-cyclic phosphate at the 3′ end (also referred to as a 3′ phosphate terminus, see FIG. 4D of Example 1). The nucleotide sequence of a target RNA molecule has at least one adenosine. In an embodiment where a target RNA has one adenosine, the sole adenosine is not the terminal nucleotide at the 3′ end. There are no other known sequence requirements, thus a target RNA may have any nucleotide sequence. Likewise, there are no requirements regarding length of a target RNA molecule. In one embodiment, a target RNA molecule is at least 19 nucleotides.

Whether a protein has A-specific RNAse activity may be determined by in vitro assays. In one embodiment, an in vitro assay is carried out as described herein (see Example 1). Briefly, a reaction can include 20 mM Tris-HCl [pH 7.5 at room temperature, pH 6.8 at 70° C.] and 100 mM NaCl with 500 nM Csx1 protein, and 15-20 fmol of target RNA for 30 minutes. In one embodiment, the temperature of the reaction when determining whether a protein has A-specific RNAse activity is 70° C. The results of the reaction may be determined by standard methods, such as electrophoretic separation using a denaturing polyacrylamide gel.

An example of a Csx1 protein is depicted at SEQ ID NO:2 (also available as Genbank accession number WP_011012267.1). Other examples of Csx1 proteins include those having sequence similarity with the amino acid sequence of SEQ ID NO:2. A Csx1 protein having sequence similarity with the amino acid sequence of SEQ ID NO:2 has A-specific RNase activity. A Csx1 protein may be isolated from a microbe, such as a member of the genera Pyrococcus, such as P. furiosus, or may be produced using recombinant techniques, or chemically or enzymatically synthesized using routine methods.

The amino acid sequence of a Csx1 protein having sequence similarity to SEQ ID NO:2 may include conservative substitutions of amino acids present in SEQ ID NO:2. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a protein. For the purposes of this invention, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids.

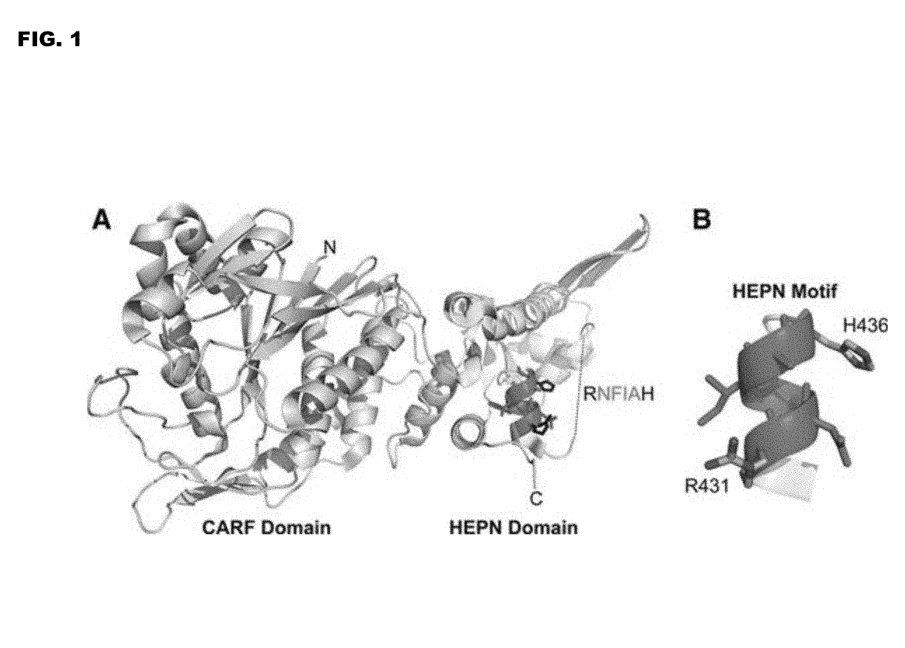

The crystal structure of a Csx1 protein having the amino acid sequence SEQ ID NO:2 has been determined (Kim et al., 2013, Proteins, 81:261-270; the P.fu amino acid sequence in FIG. 1 of Kim is SEQ ID NO:2). As shown in FIG. 1 of Kim et al., it is known that Csx1 proteins have an N-terminal domain and a C-terminal domain. The N-terminal domain includes at least one and preferably two Rossman-like folds, structural motifs made up of parallel beta strands, often found in proteins that bind nucleotides. The locations of predicted beta strands and alpha helices present in both the N-terminal and C-terminal domains are also shown in FIG. 1 of Kim et al. The N-terminal domain includes three conserved sequence motifs. The first motif is X₁X₂₋₃WGX₄X₅₋₇WX₈₋₁₁Y (SEQ ID NO:3), where X₁ is V, I, or L, X₄ is N or D, and X₂₋₃, X₅₋₇, and X₈₋₁₁ are independently any amino acid. The second motif is DX₁THGX₂NX₃X₄ (SEQ ID NO:4), where X₁ and X₂ is L, V, or I; X₃ is F or Y, and X₄ is M, L. I or V. The third motif is X₁NSX₂P (SEQ ID NO:5), where X₁ is V, Y, or L, and X₂ is E or D. The fourth motif is one diagnostic of the HEPN domain (Anantharaman et al., 2013, Biology Direct, 8:15). The fourth domain is RNX₁₋₂AHX₃G (SEQ ID NO:6), where X₁ and X₂ are independently any amino acid, and X₃ is S or A. A Csx1 protein may include 1, 2, 3, or all 4 motifs. In one embodiment, a Csx1 protein includes all 4 motifs. Based on the structural data available to the skilled person in combination with the experimental data presented herein, the skilled person can predict with a reasonable expectation of success which amino acids may be substituted, and what sorts of substitutions (e.g., conservative or non-conservative) may be made to a Csx1 protein without altering the A-specific RNAse activity of a Csx1 protein.

Further guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a protein sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

Also provided herein are isolated polynucleotides encoding a Csx1 protein. A polynucleotide encoding a protein having A-specific RNAse activity is referred to herein as a Csx1 polynucleotide. Csx1 polynucleotides may have a nucleotide sequence encoding a protein having the amino acid sequence shown in SEQ ID NO:2. An example of the class of nucleotide sequences encoding such a protein is SEQ ID NO:1. It should be understood that a polynucleotide encoding a Csx1 protein represented by SEQ ID NO:2 is not limited to the nucleotide sequence disclosed at SEQ ID NO:1, but also includes the class of polynucleotides encoding such proteins as a result of the degeneracy of the genetic code. For example, the naturally occurring nucleotide sequence SEQ ID NO:1 is but one member of the class of nucleotide sequences encoding a protein having the amino acid sequence SEQ ID NO:2. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid.

A Csx1 polynucleotide may have sequence similarity with the nucleotide sequence of SEQ ID NO:1. Csx1 polynucleotides having sequence similarity with the nucleotide sequence of SEQ ID NO:1 encode a Csx1 protein. A Csx1 polynucleotide may be isolated from a microbe, such as a member of the genera Pyrococcus, such as P. furiosus, or may be produced using recombinant techniques, or chemically or enzymatically synthesized. A Csx1 polynucleotide may further include heterologous nucleotides flanking the open reading frame encoding the Csx1 protein. Typically, heterologous nucleotides may be at the 5′ end of the coding region, at the 3′ end of the coding region, or the combination thereof. The number of heterologous nucleotides may be, for instance, at least 10, at least 100, or at least 1000.

The present invention also includes fragments of a Csx1 protein described herein and the polynucleotides encoding such fragments. A Csx1 protein fragment may include a portion of SEQ ID NO:2 or a protein having structural similarity with SEQ ID NO:2, such as a deletion of amino acids from the amino terminus, the carboxy-terminus, or a combination thereof that is at least 1, at least 2, at least 3, at least 4, at least 5, at least 6, at least 7, at least 8, at least 9, or at least 10 amino acid residues.

A Csx1 protein or fragment thereof may be expressed as a fusion protein that includes a Csx1 protein described herein and heterologous amino acids. For instance, the additional amino acid sequence may be useful for purification of the fusion protein by affinity chromatography. Amino acid sequences useful for purification can be referred to as a tag, and include but are not limited to a polyhistidine-tag (His-tag) and maltose-binding protein. Representative examples may be found in Hopp et al. (U.S. Pat. No. 4,703,004), Hopp et al. (U.S. Pat. No. 4,782,137), Sgarlato (U.S. Pat. No. 5,935,824), and Sharma Sgarlato (U.S. Pat. No. 5,594,115). Various methods are available for the addition of such affinity purification moieties to proteins. Optionally, the additional amino acid sequence, such as a His-tag, can then be cleaved.

A polynucleotide described herein may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Examples of viral vectors include, for instance, adenoviral vectors, adeno-associated viral vectors, lentiviral vectors, retroviral vectors, and herpes virus vectors. Typically, a vector is capable of replication in a microbial host, for instance, an archaeon such as P. furiosus or a bacterium such as E. coli. Preferably the vector is a plasmid.

Selection of a vector depends upon a variety of desired characteristics in the resulting construct, such as a selection marker, vector replication rate, and the like. In some aspects, suitable host cells for cloning or expressing the vectors herein include prokaryotic cells. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

Polynucleotides encoding a Csx1 protein may be obtained from microbes, for instance, members of the genus Pyrococcus, or produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are well known. Likewise, Csx1 proteins described herein may be obtained from microbes, or produced in vitro or in vivo.

An expression vector optionally includes regulatory sequences operably linked to the coding region. The invention is not limited by the use of any particular promoter, and a wide variety of promoters are known. Promoters act as regulatory signals that bind RNA polymerase in a cell to initiate transcription of a downstream (3′ direction) coding region. The promoter used may be a constitutive or an inducible promoter. It may be, but need not be, heterologous with respect to the host cell. The promoter useful in methods described herein may be, but is not limited to, a constitutive promoter, a temperature sensitive promoter, a non-regulated promoter, or an inducible promoter. A suitable promoter can cause expression of an operably linked coding region at temperatures of at least 30° C., at least 40° C., at least 50° C., at least 60° C., at least 70° C., at least 80° C., at least 90° C., or up at 100° C. A suitable promoter can cause expression of an operably linked coding region at temperatures between 30° C. and 100° C., between 50° C. and 90° C., or between 60° C. and 80° C. In one embodiment, a promoter is one that functions in a member of the domain Bacteria. In one embodiment, a promoter is one that functions in an archaeon (see Adams et al., US Patent Application Publication 2015/0211030). In one embodiment, a promoter is one that functions in a eukaryote.

An expression vector may optionally include a ribosome binding site and a start site (e.g., the codon ATG) to initiate translation of the transcribed message to produce the protein. It may also include a termination sequence to end translation. A termination sequence is typically a codon for which there exists no corresponding aminoacetyl-tRNA, thus ending protein synthesis. The polynucleotide used to transform the host cell may optionally further include a transcription termination sequence.

A vector introduced into a host cell to result in a genetically engineered archaeon optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, and neomycin. Examples of nutritional markers useful with certain host cells, including hyperthermophilic archaea and thermophilic archaea, are disclosed in Lipscomb et al. (U.S. Pat. No. 8,927,254). Examples include, but are not limited to, a requirement for uracil, histidine, or agmatine.

Proteins and fragments thereof described herein may be produced using recombinant DNA techniques, such as an expression vector present in a cell. Such methods are routine and known in the art. The proteins and fragments thereof may also be synthesized in vitro, e.g., by solid phase peptide synthetic methods. The solid phase peptide synthetic methods are routine and known in the art. A protein produced using recombinant techniques or by solid phase peptide synthetic methods may be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity.

Also provided is a genetically modified cell having a polynucleotide encoding a Csx1 protein described herein. Compared to a control cell that is not genetically modified, a genetically modified cell may exhibit production of a Csx1 protein or a fragment thereof. A polynucleotide encoding a Csx1 protein may be present in the cell as a vector or integrated into genomic DNA of the genetically modified cell, such as a chromosome or a plasmid. A cell can be a eukaryotic cell or a prokaryotic cell, such as a member of the domain Archaea or a member of the domain Bacteria

Examples of members of the domain Bacteria that can be genetically modified to include a polynucleotide encoding a Csx1 protein include, but are not limited to, Escherichia (such as Escherichia coli), Salmonella (such as Salmonella enterica, Salmonella typhi, Salmonella typhimurium), a Thermotoga spp. (such as T. maritima), an Aquifex spp (such as A. aeolicus), photosynthetic organisms including cyanobacteria (e.g., a Synechococcus spp. such as Synechococcus sp. WH8102 or, e.g., a Synechocystis spp. such as Synechocystis PCC 6803) and photosynthetic bacteria (e.g., a Rhodobacter spp. such as Rhodobacter sphaeroides), a Caldicellulosiruptor spp., such as C. bescii, and the like.

Examples of members of the domain Archaea that can be genetically modified to include a polynucleotide encoding a Csx1 protein include, but are not limited to members of the Order Thermococcales (including a member of the genus Pyrococcus, for instance P. furiosus, P. abyssi, or P. horikoshii, or a member of the genus Thermococcus, for instance, T. kodakaraensis or T. onnurineus), members of the Order Sulfolobales (including a member of the genus Metallosphaera, for instance, M. sedula), and members of the Order Thermotogales (including members of the genus Thermotoga, for instance, T. maritima or T. neapolitana). Examples of eukaryotic cells that can be genetically modified to include a polynucleotide encoding a Csx1 protein include, but are not limited to yeast such as Saccharomyces cerevisiae and Pichia spp., insect cells, and mammalian cells.

Also provided are methods for using a Csx1 protein disclosed herein. Applications of a Csx1 protein include, but are not limited to, producing an RNA molecule with a 3′-terminal adenosine; producing an RNA molecule with 5′ OH end, a 3′ phosphate end, or a combination thereof; RNA removal during DNA and/or protein isolation; RNA sequence analysis; RNase protection assays; RNA quantification or mapping (e.g., mapping cleavage sites of other ribonucleases); and isolating DNA (e.g., isolating plasmid or genomic DNA). In one embodiment, the method includes incubating a Csx1 protein and a target RNA molecule that includes a single stranded region under suitable conditions for cleavage of the target RNA molecule by the Csx1 protein. The cleavage occurs on the 3′ side of at least one adenosine residue of the target ssRNA molecule, and the cleavage results in at least one cleaved RNA molecule having an adenosine at the 3′ terminal end.

The target RNA can be from a biological sample. As used herein, a “biological sample” refers to a sample of tissue or fluid isolated from a subject, including but not limited to, for example, blood (including plasma and serum), urine, spinal fluid, lymph tissue and lymph fluid, also samples of in vitro constituents including but not limited to conditioned media resulting from the growth of eukaryotic cells, microbial cells, and tissues in culture medium. In one embodiment, a target RNA can be from an in vitro sample, for instance, RNA made by chemical or enzymatic synthesis methods.

Suitable conditions include use of a buffer having a pH of at least 6.5 to no greater than 7.5, such as 6.5, 6.6, 6.7, 6.8, 6.9, 7.0, 7.1, 7.2, 7.3, 7.4, or 7.5, at the temperature used, for instance, 70° C. The buffer may also include a salt, such as NaC1, at a concentration of, for instance, at least 50 mM to no greater than 300 mM, such as 50 mM, 75mM, 100 mM, 125, mM, 150 mM, 200 mM, 250 mM, and 300 mM. Other buffer conditions that are optional include 200 μM NiCl₂, 1.5 mM MgCl₂, 10% Glycerol, 250 mM NaCl, and 20 mM Tris-Cl, pH 7.5 (at approximately 25° C.). Optionally, a metal chelator may be included, such as EDTA at a concentration of 1 mM. In one embodiment, the temperature of an incubation may be at least 30° C., at least 40° C., at least 50° C., at least 60° C., at least 70° C., at least 80° C., or at least 90° C. In one embodiment, the temperature of an incubation may be no greater than 100° C., no greater than 90° C., no greater than 80° C., no greater than 70° C., no greater than 60° C., no greater than 50° C., or no greater than 40° C. Temperature ranges include, but are not limited to, at least 30° C. to no greater than 100° C., at least 40° C. to no greater than 90° C., and at least 50° C. to no greater than 80° C. The skilled person will recognize it is not necessary to use a Csx1 protein described herein at its optimal temperature. For instance, an optimal temperature for the Csx1 protein having the sequence SEQ ID NO:1 is 70-80° C., but it may be used at higher and lower temperatures and maintain biological activity. The skilled person will also recognize that any concentration of enzyme and any concentration of target ssRNA may be used, and that in some embodiments the target RNA used will be in an amount that yields a useful amount of product after cleavage.

Also provided is a method for making a Csx1 protein disclosed herein. In one embodiment, the method includes incubating a genetically modified cell under suitable conditions for expression of a Csx1 protein. Optionally, the method includes introducing into the cell a vector that includes a coding region encoding a Csx1 protein. In one embodiment, the method includes isolating or purifying the Csx1 protein from a cell or from a medium. In those embodiments where the Csx1 protein includes additional amino acids useful for isolating or purifying the protein, the method can also include cleavage of the additional amino acids from the Csx1 protein.

The present invention also provides a kit for cleaving a ssRNA molecule on the 3′ side of at least one adenosine residue. In one embodiment, the kit includes a Csx1 protein as described herein in a suitable packaging material in an amount sufficient for at least one assay. The Csx1 protein may be isolated or purified. In one embodiment, the kit includes a vector encoding a Csx1 protein. In one embodiment, the kit includes a genetically modified cell that includes a polynucleotide encoding a Csx1 protein. Optionally, other reagents such as buffers (either prepared or present in its constituent components, where one or more of the components may be premixed or all of the components may be separate), and the like, are also included. In one embodiment, the protein, vector, or genetically modified cell may be present with a buffer, or may be present in separate containers. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label, which indicates that the contents can be used for cleaving a single stranded RNA molecule, or used in a method that includes cleaving a single stranded RNA molecule. In addition, the packaging material contains instructions indicating how the materials within the kit are employed to cleave a single stranded RNA molecule. As used herein, the term “package” refers to a solid matrix or material such as glass, plastic, paper, foil, and the like, capable of holding within fixed limits a protein. Thus, for example, a package can include a glass or plastic vial used to contain appropriate quantities of a Csx1 protein. “Instructions for use” typically include a tangible expression describing the reagent concentration or at least one assay method parameter, such as the relative amounts of Csx1 protein and ssRNA to be admixed, maintenance time periods for reagent/sample mixtures, temperature, buffer conditions, and the like.

The present invention is illustrated by the following examples. It is to be understood that the particular examples, materials, amounts, and procedures are to be interpreted broadly in accordance with the scope and spirit of the invention as set forth herein.

### EXAMPLE 1

Prokaryotes are frequently exposed to potentially harmful invasive nucleic acids from phages, plasmids, and transposons. One method of defense is the CRISPR-Cas adaptive immune system. Diverse CRISPR-Cas systems form distinct ribonucleoprotein effector complexes that target and cleave invasive nucleic acids to provide immunity. The Type III-B Cmr effector complex has been found to target the RNA and DNA of the invader in the various bacterial and archaeal organisms where it has been characterized. Interestingly, the gene encoding the Csx1 protein is frequently located in close proximity to the Cmr1-6 genes in many genomes, implicating a role for Csx1 in Cmr function. However, evidence suggests that Csx1 is not a stably associated component of the Cmr effector complex, but is necessary for DNA silencing by the Cmr system in Sulfolobus islandicus. To investigate the function of the Csx1 protein, the activity of recombinant Pyrococcus furiosus Csx1 was characterized against various nucleic acid substrates. Csx1 is a metal-independent, endoribonuclease that acts selectively on single-stranded RNA and cleaves specifically after adenosines. The RNA cleavage activity of Csx1 is dependent upon a conserved HEPN motif located within the C-terminal domain of the protein. This motif is also relevant for activity in other known ribonucleases. Collectively, the findings indicate that invader silencing by Type III-B CRISPR-Cas systems relies both on RNA and DNA nuclease activities from the Cmr effector complex as well as on the affiliated, trans-acting Csx1 endoribonuclease.

Prokaryotes have evolved a number of ways to defend themselves from viral attack and plasmid invasion. Among these are adaptive and heritable immune systems, known as CRISPR-Cas systems, which are widespread in both bacteria and archaea (Makarova et al. 2006, Biol Direct, 1:7.; Terns et al., 2011, Curr Opin Microbiol, 14:321-7; Sorek et al., 2013, Annu Rev Biochem, 82: 237-266; van der Oost et al., 2014, Nat Rev Microbiol, 12:479-92; Jackson et al., 2015, Mol Cell, 58:722-8). CRISPR (clustered regularly interspaced short palindromic) loci contain repeat sequences that flank short DNA segments (called spacers) shown to originate from phage genomes or other invasive DNA (Bolotin et al., 2005, Microbiology, 151:2551-61; Mojica et al., 2005, J Mol Evol, 60:174-182; Pourcel et al., 2005, Microbiology, 151:653-63; Barrangou et al., 2007, Science, 315:1709-12). When foreign DNA is introduced, either by phage infection or plasmid uptake, small fragments of the invasive DNA become integrated within the CRISPR locus as a spacer (Fineran et al., 2012, Virology, 434:202-9; Nuñez et al., 2014, Nat Struct Mol Biol, 21:528-34). The primary transcript of the CRISPR locus is processed into multiple unit CRISPR RNAs (crRNAs) (Brouns et al., 2008, Science, 321:960-4; Carte et al., 2008, Genes Dev, 22:3489-96). Mature crRNAs each form ribonucleoprotein complexes with associated Cas (CRISPR-associated) proteins, and these complexes then recognize and cleave the foreign nucleic acid that is complementary to the crRNA guide element (Terns et al., 2011, Curr Opin Microbiol, 14:321-7; Westra et al., 2012, Annu Rev Genet, 46:311-39; Sorek et al., 2013, Annu Rev Biochem, 82:237-66; van der Oost et al., 2014, Nat Rev Microbiol, 12:479-92; Jackson et al., 2015, Mol Cell, 58:722-8).

CRISPR-Cas systems have been divided into five major types (I, II, III, IV, V) and at least 16 subtypes defined by the identity and arrangement of the associated cas genes and by differences in crRNA processing and invader silencing mechanisms (Makarova et al. 2011, Nat Rev Microbiol, 9:467-477; Makarova et al., 2015, Nat Rev Microbiol, 13:722-736). The hyperthermophilic archaeon Pyrococcus furiosus (Pfu) harbors three coexisting immune effector crRNP complexes: Type I-A (Csa), Type I-G (Cst), and Type III-B (Cmr), along with seven functional CRISPR loci (Hale et al. 2008, RNA, 14:2572-9; Terns et al., 2013, Biochem Soc Trans, 41:1416-21; Majumdar et al., 2015, RNA, 21:1147-58). There is evidence that the Pfu Csa and Cst effector complexes target DNA (Elmore et al., 2015, Nucleic Acids Res, 43:10353-63), while the Cmr complex has been shown to target DNA and RNA in vitro and in vivo (Hale et al., 2009, Cell, 139:945-56; Hale et al., 2012, Mol Cell, 45:292-302; Hale et al., 2014, Genes Dev 28: 2432-43; Deng et al., 2013, Mol Microbiol, 87:1088-99; Spilman et al., 2013, Mol Cell, 52:146-52; Benda et al. 2014, Mol Cell, 56:43-54; Ramia et al., 2014, Cell Rep, 9:1610-7).

The Pfu Cmr RNA-targeting mechanism and necessary components have recently been characterized. The Cmr complex consists of Cmr1-6 proteins in association with a single crRNA (Hale et al., 2009, Cell, 139:945-56; Spilman et al., 2013, Mol Cell, 52:146-52). The interaction of the Cmr complex with target RNA is guided by crRNA/target RNA complementary base-pairing (Hale et al., 2009, Cell, 139:945-56; Hale et al., 2012, Mol Cell, 45:292-302; Hale et al., 2014, Genes Dev 28: 2432-43; Ramia et al., 2014, Cell Rep, 9:1610-7). Multiple Cmr4 subunits, which form the backbone of the complex, mediate cleavage of the bound target RNA at regular 6-nt intervals (Staals et al., 2013, Mol Cell, 52:135-45; Benda et al. 2014, Mol Cell, 56:43-54; Hale et al., 2014, Genes Dev 28: 2432-43; Ramia et al., 2014, Cell Rep, 9:1610-7; Taylor et al., 2015, Science, 348:581-5). Recent data indicate that the Cmr system of Sulfolobus islandicus is capable of transcription-dependent, plasmid silencing in vivo, although this activity has not been recreated with purified components or characterized in detail (Deng et al., 2013, Mol Microbiol, 87:1088-99).

Notably, the csx1 gene is tightly evolutionarily linked with Type III CRISPR-Cas systems (Garrett et al., 2011, Trends Microbiol, 19: 549-56; Makarova et al. 2011, Nat Rev Microbiol, 9: 467-477; Makarova et al., 2013, Evolution and classification of CRISPR-Cas systems and cas protein families, In: CRISPR-Cas systems (eds. Barrangou et al.,), pp. 61-91. Springer, Berlin/Heidelberg). In Pfu, the csx1 (PF1127) gene is located between the cmr3 (PF1128) and cmr4 (PF1126) genes (Terns et al., 2013, Biochem Soc Trans, 41:1416-21). However, data from in vitro and in vivo assays indicate that Pfu Csx1 is not necessary for Cmr-mediated RNA or DNA targeting (Hale et al., 2009, Cell, 139:945-56; Hale et al., 2012, Mol Cell, 45:292-302; Hale et al., 2014, Genes Dev 28: 2432-43; Spilman et al., 2013, Mol Cell, 52:146-52). On the other hand, in S. islandicus, Csx1 was shown to be necessary for Cmr-mediated, transcription-dependent plasmid silencing in vivo, although the specific role of the Csx1 protein is unknown (Deng et al., 2013, Mol Microbiol, 87:1088-99).

The crystal structure of Pfu Csx1 was determined (Kim et al., 2013, Proteins, 81: 261-70), revealing an elongated structure with clearly identifiable N- and C-terminal domains. The N-terminal domain is composed of two Rossmann-like folds, while the C-terminal domain exhibits reported structural similarity to a winged-helix domain (FIG. 1A). Amino acid sequence alignments of Csx1 homologs reveals that the N-terminal domain is relatively well conserved, while there is minimal homology in the C-terminal domain, except for one short motif, R—X4-6—H, that is diagnostic of the HEPN (higher eukaryotes and prokaryotes nucleotide-binding) domain (FIG. 1B; Anantharaman et al., 2013, Biol Direct, 8:15). While the HEPN domain was originally identified as being fused or associated with a nearby nucleotidyl transferase domain (Grynberg et al., 2003, Trends Biochem Sci, 28:224-6), the HEPN protein superfamily was recently expanded to encompass proteins linked to prokaryotic viral defense systems, including the Type III CRISPR-Cas-associated Csx1 and Csm6 proteins (which belong to the COG1517 superfamily), as well as a number of predicted ribonucleases from toxin/antitoxin (T-A) modules and abortive infection (Abi) systems (Makarova et al., 2012, Biol Direct, 7:40; Makarova et al., 2014, Front Genet, 5:102; Anantharaman et al., 2013, Biol Direct, 8:15).

The N-terminal Rossmann fold is a unifying feature of a recently proposed family of proteins with largely undefined functions termed CARF (CRISPR-associated Rossmann fold) proteins (Makarova et al., 2014, Front Genet, 5:102). As Rossmann folds are known (di)nucleotide-binding domains, CARF proteins have been predicted to act as ligand-controlled transcriptional regulators of CRISPR-Cas systems and/or active components of cell defense mechanisms (Lintner et al., 2011, J Mol Biol, 405: 939-55; Makarova et al., 2012, Biol Direct, 7:40, Makarova et al., 2014, Front Genet, 5:102; Anantharaman et al., 2013, Biol Direct, 8:15; Liu et al., 2015, Nucleic Acids Res, 43:1044-55). Pfu Csx1 was reported to bind double-stranded RNA and DNA in vitro in a sequence-independent manner, although no nucleic acid cleavage activity was reported (Kim et al., 2013, Proteins, 81: 261-70). The activity of Pfu Csx1 in vitro is investigated and shows to be a single-strand-specific endoribonuclease that cleaves specifically after adenosines.

### Materials and Methods

### Purification of Csx1

The gene encoding P. furiosus Csx1 (PF1127) was amplified by PCR from genomic DNA and cloned into a modified form of pET24d. N-terminal, 6x-histidine-tagged Csx1 protein was expressed in Escherichia coli BL21-RIPL cells (DE3, Stratagene). Cells (1 L culture) were grown to an OD₆₀₀ of 0.7, and protein expression was induced overnight at room temperature by the addition of isopropylthio-β-D galactoside (IPTG) to a final concentration of 1 mM. The cells were resuspended in native binding buffer (NBB; 50 mM sodium phosphate [pH 7.6], 500 mM NaCl, and 0.1 mM phenylmethylsulfonyl fluoride) and were disrupted by sonication (Misonix Sonicator 3000). The lysate was cleared by centrifugation at 6000 rpm for 10 min, followed by incubation at 70° C. for 20 min. The sample was centrifuged at 9000 rpm for 10 min, syringe-filtered (Corning Incorporated, 0.80 μm), and applied to a HisTrap HP column (GE Healthcare) that had been equilibrated with NBB. The protein was eluted from the column using NBB containing increasing concentrations of imidazole (50, 100, 200, and 500 mM). Fractions were evaluated by SDS-PAGE and staining with Coomassie blue. The peak fraction of Csx1 was further purified by gel filtration using an XK26 HiLoad 26/60 Superdex 200 gel filtration column (GE Healthcare) that had been equilibrated with 2× assay buffer (40 mM Tris-HCl [pH 7.5] and 200 mM NaCl).

Generation of RNA and DNA ubstrates

Synthetic RNAs were purchased from Integrated DNA Technologies (IDT), DNA oligos from Eurofins MWG Operon, and the RNA size standards (Decade Markers) from Life Technologies. The sequences of the RNAs used in this study are given in Table 1. The oligonucleotides were 5′ end-labeled with T4 polynucleotide kinase (New England Biolabs [NEB]) in a 20 μL reaction containing 20 pmol oligonucleotide, 150 μCi of [γ-32P] ATP (6000 Ci/mmol; Perkin Elmer), 1×T4 PNK buffer, and 10 U of T4 kinase (NEB). RNAs were 3′ end-labeled with T4 RNA ligase (NEB) in a 20 μL reaction containing 20 pmol RNA, 10 μCi of [α-32P] pCp (3000 Ci/mmol; Perkin Elmer), 20 U of T4 ligase, 10 U of SUPERase-IN RNase inhibitor (Ambion), 1×T4 RNA ligase buffer (NEB), and 20% polyethylene glycol M.W. 8000 (NEB). The oligonucleotides were then run on a denaturing (7 M urea) 15% polyacrylamide gel containing 1×TBE (89 mM Tris base, 89 mM Boric acid, 2 mM EDTA, pH 8.0), followed by autoradiographic exposure to guide excision of the appropriate bands. The oligonucleotides were eluted by end-over-end rotation for 12-14 h at 4° C. in 500 μL of 2× assay buffer. This was followed by phenol/chloroform/isoamyl alcohol (PCI, 25:24:1 at pH 5.2; Fisher Biosciences) extraction, then precipitation with 2.5 volumes of 100% ethanol, 0.3 M sodium acetate, and 20 μg glycogen after incubation for 30 min at −80° C.

TABLE 1
Sequences of RNA and DNA substrates.

| RNA | Sequence (5′-3′) |
|---|---|
| 37 mer A | CUGAAGUGCUCUCAGCCGCAAGGACCGCAUACUACAA (SEQ ID NO: 3) |
| 37 mer B | UUGUAGUAUGCGGUCCUUGCGGCUGAGAGCACUUCAG (SEQ ID NO: 4) |
| 45 mer A | AUUGAAAGUUGUAGUAUGCGGUCCUUGCGGCUGAGAGCACUUCAG (SEQ ID |
| | NO: 5) |
| 45 mer B | AUUGAAAGAGGGAAUAAGGGCGACACGGAAAUGUUGAAUACUCAU (SEQ ID |
| | NO: 6) |
| 45 mer C | AUUGAAAGAGUGAAGAAUUUGACGUACAAAUGUCCUUAGUGGAAC (SEQ ID |
| | NO: 7) |
| 67 mer | AUUGAAAGUUGUAGUAUGCGGUCCUUGCGGCUGAGAGCACUUCAGUCGUU |
| | AUCUCUUACGAAGUCUU (SEQ ID NO: 8) |
| poly(A) | AAAAAAAAAAAAAAAAAAA (SEQ ID NO: 9) |
| poly(G) | GGGGGGGGGGGGGGGGGG (SEQ ID NO: 10) |
| poly(U) | UUUUUUUUUUUUUUUUUUU (SEQ ID NO: 11) |
| poly(C₁₀) (AUG)₃ | CCCCCCCCCCAUGAUGAUG (SEQ ID NO: 12) |
| DNA | Sequence (5′-3′) |
| 63 mer A | ATTTAGGTGACACTATAGATTGAAAGTTGTAGTATGCGGTCCTTGCGGCTGAG |
| | AGCACTTCAG (SEQ ID NO: 13) |
| 63 mer B | CTGAAGTGCTCTCAGCCGCAAGGACCGCATACTACAACTTTCAATCTATAGTG |
| | TCACCTAAAT (SEQ ID NO: 14) |
| 45 mer D | CTGAAGTGCTCTCAGCCGCAAGGACCGCATACTACAACTTTCAAT (SEQ ID |
| | NO: 15) |

Double-stranded oligonucleotides were created by mixing labeled oligonucleotides with a twofold molar excess of nonlabeled complement in 30 mM HEPES (pH 7.4), 100 mM potassium acetate, 2 mM magnesium acetate and incubating for 1 min at 95° C., followed by temperatures decreasing by 1° each minute, down to 23° C. Annealing was confirmed and substrates were purified following electrophoresis on nondenaturing 15% polyacrylamide gels. Double-stranded substrates were then removed, eluted, extracted, and precipitated as described above, but PCI of pH 8.0 was used.

Circular RNAs were created using 5′ end-labeled RNA (67mer A), as described above, in a 20 μL reaction containing ˜10 pmol RNA, 20 μg BSA, 1 mM ATP, 20 U of T4 ligase, 10 U of SUPERase-IN RNase inhibitor, and 1×T4 RNA ligase buffer. Circularization was confirmed and circular RNA was purified with denaturing (8.3 M urea) 20% polyacrylamide gels in TBE. The circular RNA was then removed, eluted, extracted, and precipitated as described above.

### Nuclease Assays

Assays were carried out in 20 μL reactions made up of 1× assay buffer (20 mM Tris-HCl [pH 7.5 at room temperature] and 100 mM NaCl) with 500 nM Csx1, as determined by Qubit 2.0 Fluorometer (Life Technologies) quantification, and 5000 cpm (˜15-20 fmol) of oligonucleotide at 70° C. for 30 min, unless otherwise noted in Results. Assays involving double-stranded nucleic acids were incubated at 60° C. to reduce heat-induced strand separation. Reactions were stopped by placing tubes on ice and adding an equal volume of Gel Loading Buffer II (95% formamide, 18 mM EDTA, and 0.025% SDS, Xylene Cyanol, and Bromophenol Blue; Life Technologies). The reaction products were separated by electrophoresis on either 15% (7.0 M urea, linear substrates) or 20% (8.3 M urea, circular RNAs) denaturing polyacrylamide gels. Radiolabeled Decade Markers (Life Technologies) were used to determine the sizes of observed products. For sequencing gels, partial alkaline hydrolysis (cleaves phosphodiester linkages) and RNase T1 (cleaves after guanylate residues) ladders (Ambion) were generated using single-hit conditions, as described by the manufacturer. Gels were dried, and radiolabeled substrates were visualized by phosphorimaging.

### Creation of Csx1 Mutants

QuikChange site-directed mutagenesis (Stratagene) was used to create site-specific mutations in the csx1 gene. The R431A mutant was generated using the primers 5′-gacaatagaatctccaaatgttgttgctaactttatagcacattctggattt (SEQ ID NO:16) and 5′-aaatccagaatgtgctataaagttagcaacaacatttggagattctattgtc (SEQ ID NO:17). The H436A mutant was generated using the primers 5′ caaatgttgttcgtaactttatagcagcttctggatttgagtataacattgtct (SEQ ID NO:18) and 5′-agacaatgttatactcaaatccagaagctgctataaagttacgaacaacatttg (SEQ ID NO:19). The R431A+H436A double mutant was generated using primers 5′-gacaatagaatctccaaatgttgttgctaactttatagcagcttctggattt (SEQ ID NO:20) and 5′-aaatccagaagctgctataaagttagcaacaacatttggagattctattgtc (SEQ ID NO:21) using the plasmid encoding the R431A csx1 mutant gene. Mutations were confirmed by sequencing. The mutant proteins were expressed as described above and purified using a Ni-NTA agarose column (Qiagen).

### End-Group Analysis for Cleaved RNA

Circular, 5′ end-labeled, and 3′ end-labeled RNAs were treated with Csx1, as described above. Products of circular and 3′ end-labeled RNA were treated with 1 U Terminator Exonuclease (TEX; EpiBio), 1× terminator reaction buffer B (EpiBio), and 10 U of SUPERase-IN RNase inhibitor and incubated at 42° C. for 30 min. Products of 5′ end-labeled RNA were treated with 5 U E. coli poly(A) polymerase (PAP; NEB), 1× PAP reaction buffer (NEB), and 10 U of SUPERase-IN RNase inhibitor and incubated at 37° C. for 20 min. Reactions were stopped by placing on ice and adding an equal volume of Gel Loading Buffer II (Life Technologies). The reaction products were separated by electrophoresis on denaturing 15% or 20% polyacrylamide as described above

### Results

### Csx1 Cleaves Single-Stranded RNA

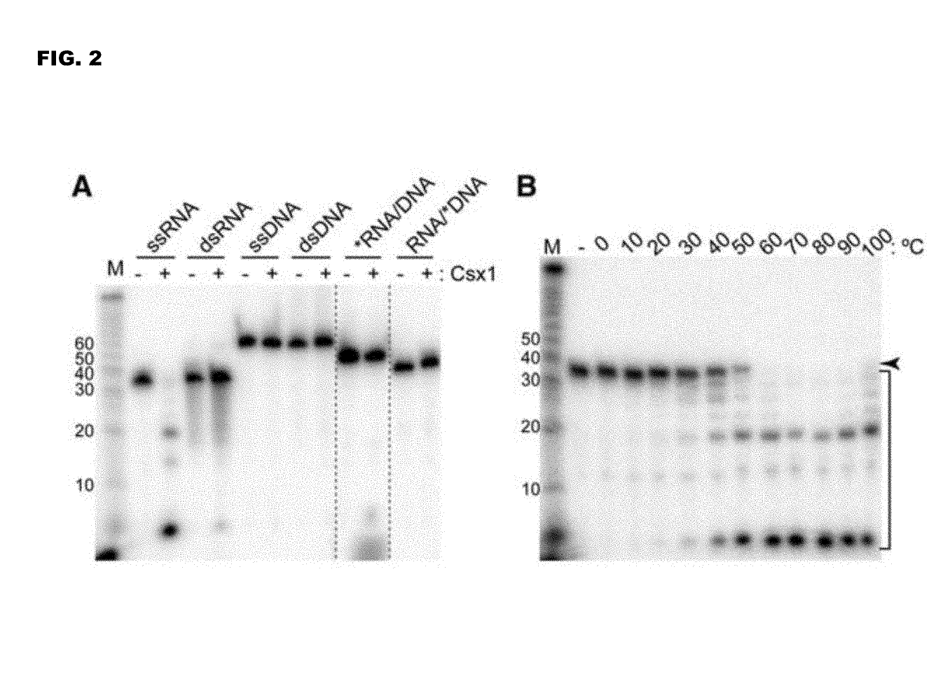

CRISPR-Cas systems rely on various nucleases to cleave RNA or DNA targets. To determine if Csx1 is a nuclease, 5′-radiolabeled single-stranded RNA (ssRNA, 37mer A), double-stranded RNA (dsRNA, 37mers A+B), ssDNA (63mer A), dsDNA (63mers A+B), and an RNA/DNA hybrid (45mers A+D) were treated with purified recombinant His-tagged Csx1 (FIG. 2A and see Table 1 for sequences of the nucleic acids used in this and all other experiments). The ssRNA was efficiently cleaved, but none of the other substrates showed significant cleavage, and no cleavage was observed in the absence of Csx1. The small amount of dsRNA and RNA/DNA hybrid cleavage observed is likely due to limited formation of ssRNA in these samples caused by strand separation during incubation at 60° C. The results indicate that recombinant Csx1 has cleavage activity that is specific for ssRNA.

Proteins from hyperthermophiles, like Pfu, typically function optimally at elevated temperatures. The optimal temperature for ssRNA cleavage by the Csx1 enzyme was determined by performing the reaction across a wide range of temperatures (FIG. 2B). This analysis showed that Csx1 performs optimally at or above 60° C. and was highly active even at 100° C. Under conditions where almost all of the full-length input ssRNA (37mer A) was cleaved, shorter cleavage products persisted, suggesting that Csx1 has a limited substrate specificity. Previous work by others had shown that specific mutations in the conserved HEPN motif (R—X₄₋₆—H, where X is any amino acid) of other known ribonucleases abolished or abrogated the cleavage activity, indicating that this highly conserved motif acted as an RNase active site. Specifically, it was shown that mutation of the conserved histidine eliminates the RNase activity of bacterial antiviral tRNA ribonucleases PrrC (Meineke et al. 2011, Nucleic Acids Res, 39:687-700; Meineke et al., 2012, Virology 427:144-50) and RloC (Davidov et al., 2008, Mol Microbiol 69: 1560-74), as well as eukaryotic Ire1 and antiviral RNase L (Dong et al., 2001, RNA, 7:361-73; Lee et al., 2008, Cell, 132:89-100; Han et al., 2014, Science 343:1244-8). Mutating the conserved arginine of PrrC (Meineke et al. 2011, Nucleic Acids Res, 39:687-700) or Ire1 (Dong et al., 2001, RNA, 7:361-73) also blocks catalytic activity.

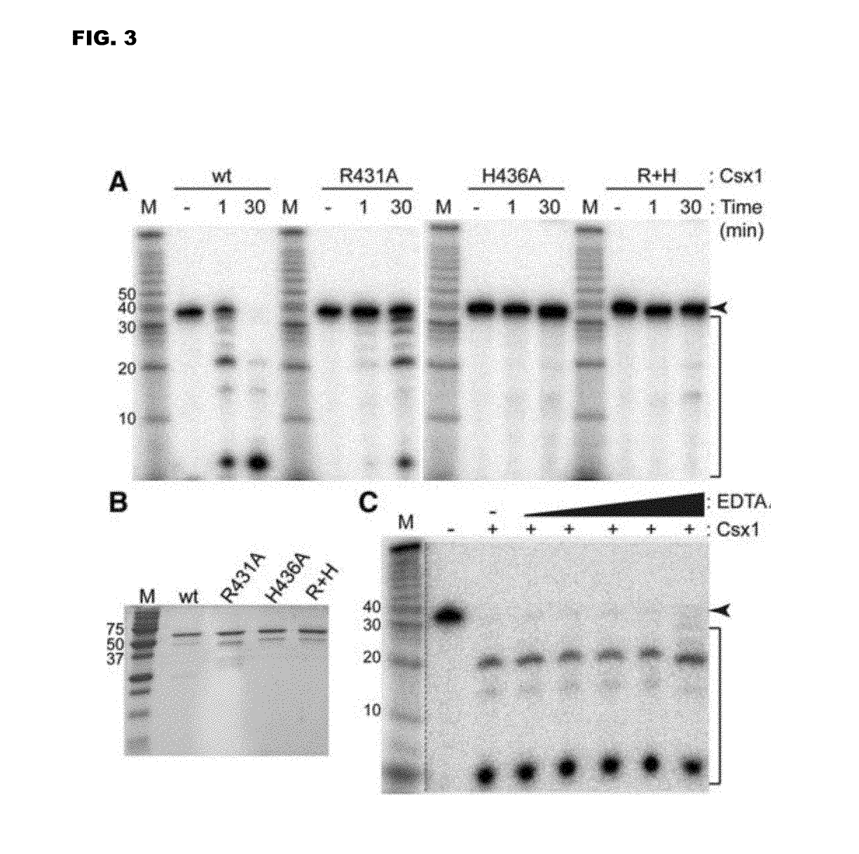

We tested the prediction that the conserved motif present in the C-terminal domain of Csx1 proteins is responsible for the RNA cleavage activity of Csx1 by mutating the highly conserved residues (R431A and H436A) individually, as well as in combination (FIG. 3A). An equal concentration of wild-type or mutant Csx1 (FIG. 3B) was used in a reaction with ssRNA (37mer A), with time points taken at 1 min and 30 min (FIG. 3A). A similar cleavage pattern was observed for both wild-type and R431A Csx1 mutant; however, the rate of cleavage was significantly reduced for the mutant protein (note that at the 30 minute time point, nearly all RNA was cleaved by the wild-type protein, but only a small fraction was cleaved by the mutant protein). In contrast, the activity of the Csx1 protein was abolished by H436A and R431A+H436A mutations. These observations suggest that the conserved HEPN-associated, R—X₄₋₆—H motif found in the C-terminal domain, is relevant for the ribonuclease activity of Csx1.

### Cleavage Mechanism

The ssRNA cleavage activity of Csx1 appears to be metal ion-independent. The metal independence of the reaction is supported by the observation that RNA cleavage by Csx1 occurs in the absence of added metals in the reaction buffer (FIG. 3A,C). Moreover, the RNA cleavage activity of Csx1 is unaffected by the addition of up to millimolar concentrations of the divalent metal ion chelator EDTA (FIG. 3C). Other characterized HEPN RNases employ a metal ion-independent catalytic mechanism (Anantharaman et al., 2013, Biol Direct, 8:15).

To determine whether Csx1 acts as an exo- or endoribonuclease, we tested whether Csx1 could cleave circular RNAs, as would be expected for an endonuclease but not exonuclease (FIG. 4A). 5′-Radiolabeled ssRNA (67mer) was circularized and treated with Csx1, with time points taken at 1 and 30 min. Terminator 5′-phosphate-dependent exonuclease (TEX), which cleaves RNA with a 5′ phosphate, was used to determine the success of circularization. The linear radiolabeled control RNA was cleaved by TEX as expected, while the circular RNA remained intact (FIG. 4A). After 1 min, the circular substrate exhibited a cleavage product the same size as the full-length linear RNA, suggesting a single cleavage by Csx1. Smaller cleavage products were observed in lower abundance. After 30 min, the input RNA was fully cleaved. Due to the radiolabel on the circular RNA becoming internal, different cleavage products are observed with the circular RNA as compared to the linear RNA. These results indicate that Csx1 acts as an endoribonuclease.

Next, we mapped the 5′ and 3′ end groups of the RNA cleavage products generated by Csx1 cutting (FIG. 4B,C). To this end, 5′-radiolabeled ssRNA (45mer A) was treated with or without Csx1 under reaction conditions that did not go to completion and thus retained some of the uncleaved, full-length RNA species. The RNA products were then treated with poly(A) polymerase (PAP), which adds poly(A) stretches to RNAs with 3′ OH groups (FIG. 4B). In the absence of Csx1 treatment, the full-length RNA was extended by PAP as expected. When incubated in the presence of Csx1, the full-length (uncleaved) RNA in the sample was extended, while the Csx1-generated RNA cleavage products were not extended. This result indicates that the 3′ ends produced by Csx1 cleavage lack a 3′ OH group.

To determine the 5′ end group of Csx1 cleavage products, 3′-radiolabeled ssRNA (45mer A) was treated as described above. The RNA was treated with TEX (5′-3′ exonuclease that selectively digests RNA having a 5′ monophosphate end) to test for the presence of 5′ phosphates on the Csx1 cleavage products (FIG. 4C). Both the full-length RNA and cleavage products were resistant to TEX degradation, while the 5′-radiolabeled control RNA was successfully cleaved as expected. This result indicates that Csx1 cleavage does not result in cleavage products containing 5′ phosphates. Taken together, these data are consistent with Csx1 being a metal-independent endoribonuclease leaving cleavage products with a 5′ OH group and 2′, 3′-cyclic phosphate or 3′ phosphate termini (FIG. 4D; Yang, 2011, Q Rev Biophys, 44:1-93).

### Sequence Specificity

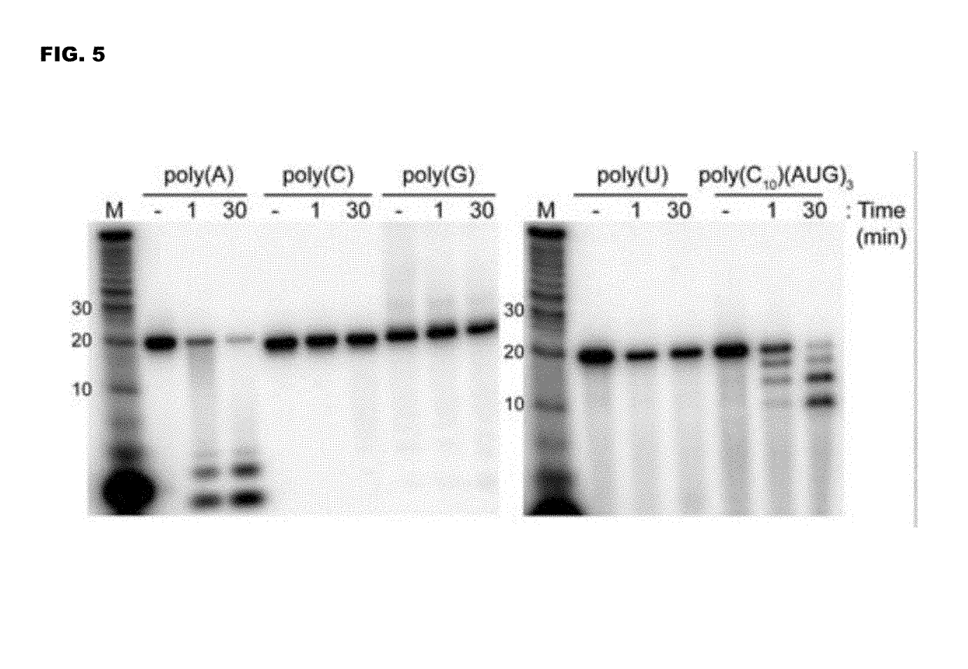

To investigate whether Csx1 cleavage activity had any sequence specificity, we treated all possible RNA homoribopolymers [poly(A), poly(C), poly(G), poly(U)], as well as a poly(C₁₀)/(AUG)₃ RNA with Csx1 (FIG. 5 and see Table 1 for sequences of the RNA substrates). We observed robust cleavage of the poly(A) RNA, but no cleavage of the other homoribopolymers. We also observed three products from cleavage of the poly(C₁₀)/(AUG)₃ RNA, with sizes consistent with cleavage after each adenosine in the RNA.

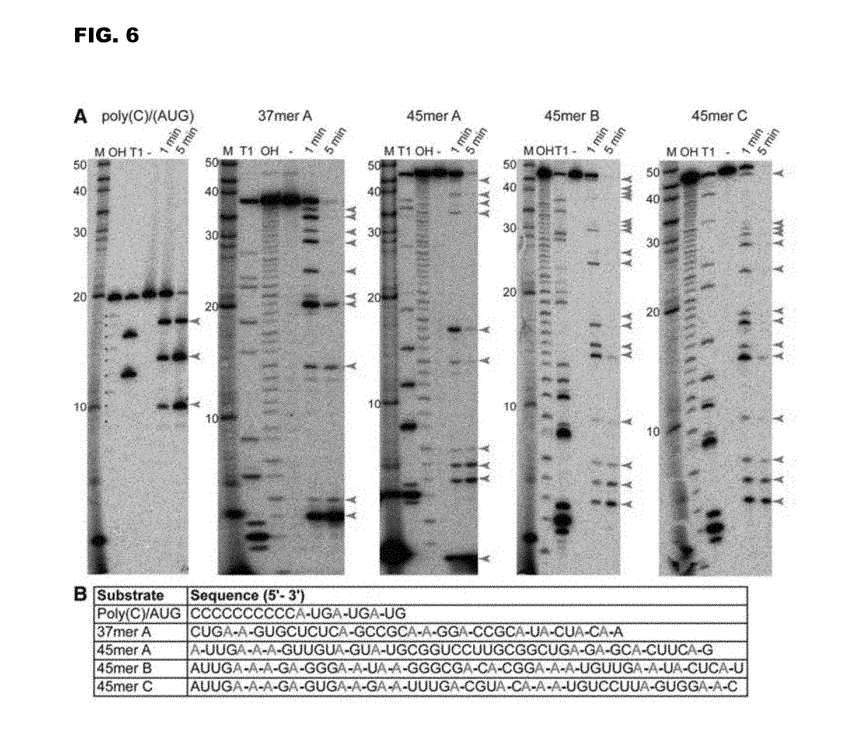

To get a clearer picture of this apparent base specificity, we treated four “mixed-sequence” RNAs and the poly(C₁₀)/(AUG)₃ RNA with Csx1 (FIG. 6A). These were run on sequencing gels, and the cleavage products were mapped at nucleotide resolution to the sequences (FIG. 6B). Alkaline hydrolysis and RNase T1 ladders of each substrate RNA were used in parallel to determine sites of Csx1 cleavage. This mapping revealed that Csx1 cleaved each of the input substrate RNAs after every adenosine in the RNA and not after any other nucleotide.

### Discussion

Despite its prevalent association with Type III CRISPR-Cas systems (Haft et al., 2005, PLoS Comput Biol, 1:e60; Garrett et al., 2011, Trends Microbiol, 19: 549-56; Makarova et al. 2011, Nat Rev Microbiol, 9: 467-477), the function and activity of Csx1 proteins have remained largely uncharacterized. Here we have experimentally determined that Pfu Csx1 functions as a metal-independent, single-strand-specific endoribonuclease that relies on an HEPN active site found in other characterized RNases (Dong et al., 2001, RNA, 7:361-73; Davidov et al., 2008, Mol Microbiol 69: 1560-74; Lee et al., 2008, Cell, 132:89-100; Meineke et al. 2011, Nucleic Acids Res, 39:687-700; Meineke et al., 2012, Virology 427:144-50; Anantharaman et al., 2013, Biol Direct, 8:15). The RNase activity of Csx1 was previously anticipated based on the occurrence of the highly conserved HEPN motif in Csx1 homologs by sequence analysis (Makarova et al., 2012, Biol Direct, 7:40; Anantharaman et al., 2013, Biol Direct, 8:15).

Interestingly, we found that Pfu Csx1 cleaves specifically after adenosines (FIGS. 5, 6). An RNase with complete specificity for adenosines has not been reported. While the RNases T2 and U2 have been shown to have a preference for adenosines, they have also been found to cleave after other nucleotides, and U2 cleavage is highly dependent on the adjacent nucleotides (Rogg et al., 1972, Biochim Biophys Acta, 262:314-9; Yasuda et al., 1982, Biochemistry, 21: 364-9; Deshpande et al., 2002, Crit Rev Microbiol, 28:79-122; Macintosh, 2011, RNase T2 family: enzymatic properties, functional diversity, and evolution of ancient ribonucleases, In: Ribonucleases (ed. Nicholson), pp. 89-114. Springer, Berlin/Heidelberg.). In contrast, Pf Csx1 shows remarkable specificity for cleaving diverse RNA substrates at sites containing an adenosine in several sequence contexts (FIG. 6). The novel specificity of Pf Csx1 as an adenosine-specific RNA cleaving enzyme has the potential to be leveraged as a useful molecular tool. Analogous to the commonly used RNase T1 enzyme that specifically cleaves RNAs after guanine (Sato et al., 1957, J Biochem, 44:753-67), Csx1 has the potential to be used in determining RNA sequence, mapping cleavage sites of other ribonucleases, and leaving RNAs with 3′-terminal adenosines, among other potentially useful applications.

Our mutational analysis of the HEPN R—X4-6—H motif of Csx1 confirms that the highly conserved arginine and histidine are important for RNase activity (as shown with other studied HEPN RNases) and provides insight into the possible catalytic mechanism of the enzyme (FIG. 3; Dong et al., 2001, RNA, 7:361-73; Davidov et al., 2008, Mol Microbiol 69: 1560-74; Lee et al., 2008, Cell, 132:89-100; Meineke et al. 2011, Nucleic Acids Res, 39:687-700; Meineke et al., 2012, Virology 427:144-50; Anantharaman et al., 2013, Biol Direct, 8:15). Consistent with findings for other HEPN RNases (Anantharaman et al., 2013, Biol Direct, 8:15), our results support a metal ion-independent cleavage mechanism for Csx1, generating RNA fragments with 5′ hydroxyl and 2′,3′-cyclic phosphate termini (FIGS. 3, 4). Based on the proposed general acid-base catalytic mechanism of other HEPN RNases (Anantharaman et al., 2013, Biol Direct, 8:15), the predicted Csx1 active site His436 likely functions as a general base to deprotonate the nucleophilic 2′-hydroxyl of the ribose ring leading to an attack of the 2′ oxygen on the phosphate backbone. Alternatively or additionally, His436 may act as a general acid to protonate the 5′ oxyanion leaving group to facilitate cleavage of the scissile phosphate. We found that mutation of Csx1 His436 abolished activity, while mutation of the predicted active site Arg431 residue significantly impaired, but did not prevent, RNA cleavage by the Csx1 enzyme (FIG. 3). The role of the arginine may be charge stabilization of the predicted pentavalent transition state during the cleavage reaction or interaction with the backbone of the RNA substrate. A Csx1-specific HEPN motif consensus motif was determined as R—N—X-θ-A-H (Kim et al., 2013, Proteins, 81: 261-70), suggesting that the identity of the residues flanking the broadly conserved R and H residues may also be important for Csx1 activity.

Csx1 is structurally related to the Csm6 protein, and, by inference, our results make a strong prediction that Csm6 also exhibits single-strand-specific RNase activity. Indeed, the many shared features of Csx1 and Csm6 indicate that these proteins perform similar or identical functional roles. Csx1 and Csm6 are each CARF proteins that harbor N-terminal Rossman fold domains and C-terminal domains containing the R-X₄.₆-H HEPN RNase active site (Makarova et al., 2012, Biol Direct, 7:40, Makarova et al., 2014, Front Genet, 5:102; Anantharaman et al., 2013, Biol Direct, 8:15). The csx1 and csm6 genes are evolutionarily linked to Type III-B (Cmr) and Type III-A (Csm) CRISPR-Cas systems, respectively (Garrett et al., 2011, Trends Microbiol, 19: 549-56; Makarova et al., 2013, Evolution and classification of CRISPR-Cas systems and cas protein families, In: CRISPR-Cas systems (eds. Barrangou et al.,), pp. 61-91. Springer, Berlin/Heidelberg), indicating these two protein families cofunction with Type III CRISPR-Cas systems, which are known to cleave both target (e.g., viral) RNA as well as target DNA in a transcription-dependent manner (Hale et al., 2009, Cell, 139:945-56; Marraffini et al., 2010, Nature, 463:568-71; Zhang et al., 2012, Mol Cell, 45:303-13; Deng et al., 2013, Mol Microbiol, 87:1088-99; Staals et al., 2013, Mol Cell, 52:135-45; Staals et al., 2014, Mol Cell, 56:518-30; Hale et al., 2014, Genes Dev 28: 2432-43; Hatoum-Aslan et al., 2014, J Bacteriol, 196:310-7; Ramia et al., 2014, Cell Rep, 9:1610-7; Tamulaitis et al., 2014, Mol Cell, 56: 506-17; Samai et al., 2015, Cell, 161:1164-74).

The function of Csx1 and Csm6 Cas proteins remains enigmatic. Intriguingly, evidence has emerged that both csx1 and csm6 genes are vital for transcription-dependent plasmid interference in vivo (Deng et al., 2013, Mol Microbiol, 87:1088-99; Hatoum-Aslan et al., 2014, J Bacteriol, 196:310-7), despite clear evidence in vitro that both Csx1 and Csm6 proteins are dispensible for target RNA cleavage (Hale et al., 2009, Cell, 139:945-56; Hale et al., 2014, Genes Dev 28: 2432-43; Zhang et al., 2012, Mol Cell, 45:303-13; Staals et al., 2013, Mol Cell, 52:135-45; Staals et al., 2014, Mol Cell, 56:518-30; Ramia et al., 2014, Cell Rep, 9:1610-7; Tamulaitis et al., 2014, Mol Cell, 56: 506-17; Samai et al., 2015, Cell, 161:1164-74) as well as for transcription-dependent target DNA cleavage (Samai et al., 2015, Cell, 161:1164-74). Furthermore, Csx1 and Csm6 are not required for the proper processing or maturation of crRNAs (Hatoum-Aslan et al., 2014, J Bacteriol, 196:310-7), and neither protein is stably associated with its affiliated multisubunit Cmr or Csm crRNP effector complex, respectively (Hale et al., 2009, Cell, 139:945-56; Hatoum-Aslan et al., 2014, J Bacteriol, 196:310-7). These observations indicate that Csx1 and Csm6 may play a role in antiviral defense that is auxiliary to that of the evolutionarily linked Cmr and Csm effector crRNPs.

Our results indicate a possible key role for RNase activity in the functioning of Csx1 and Csm6 CARF proteins. Conceivably, Csx1 and Csm6 are regulated to selectively destroy invasive RNAs (e.g., viral mRNAs) either in addition to, or in conjunction with, the crRNP-guided Type III effector complexes. Another intriguing proposal is that these CARF proteins may cleave (certain) host RNAs to act as dormancy/suicide inducers in the event the CRISPR defense mechanism fails to dispel the invader in a timely manner (Makarova et al., 2012, Biol Direct, 7:40; Anantharaman et al., 2013, Biol Direct, 8:15). It is not clear how Csx1 or Csm6 RNase activity might affect transcription-dependent DNA silencing activity of Cmr and Csm effector complexes or whether the observed adenosine-specific cleavage by Csx1 (FIGS. 5, 6) is significant for its physiological function.

Understanding how Csx1 (and related Csm6) activity is regulated remains an important challenge. In general, the activity of cellular ribonucleases is tightly controlled such that they cleave only their intended substrates. We have found that Csx1 protein is constitutively expressed in Pfu cells, suggesting that Csx1 activity may be post-translationally controlled in vivo. Indeed, the N-terminal CARF domain of Csx1 (Kim et al., 2013, Proteins, 81: 261-70) is predicted to interact with a yet-to-be-determined (di)nucleotide that may allosterically regulate Csx1 cleavage activity, perhaps in response to viral infection and associated nucleotide metabolites that might be triggered in response to the invasion (Lintner et al., 2011, J Mol Biol 405: 939-955; Makarova et al., 2012, Biol Direct, 7:40, Makarova et al., 2014, Front Genet, 5:102; Anantharaman et al., 2013, Biol Direct, 8:15). The oligomeric state of the protein may represent an additional point of control for the activity of Csx1 (and Csm6). Monomeric Pfu Csx1 was found to homodimerize following binding to dsDNA, bringing the HEPN RNase active sites in close proximity to one another (Kim et al., 2013, Proteins, 81: 261-70). This raises the possibility that there is a nucleic acid regulator of Csx1 function.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
incubating a sample comprising an isolated Csx1 protein and a target RNA molecule comprising a single stranded region under suitable conditions for cleavage of the target RNA molecule by the Csx1 protein, wherein the cleavage occurs on the 3′ side of at least one adenosine residue of the target RNA molecule, and wherein the cleavage results in at least one cleaved RNA molecule comprising an adenosine at the 3′ terminal end.

2. The method of claim 1 wherein the target RNA molecule is a single stranded RNA molecule.

3. The method of claim 1 wherein the target RNA molecule is linear.

4. The method of claim 1 wherein the target RNA molecule is from a biological sample.

5. The method of claim 4 wherein the biological sample is from a microbial cell.

6. The method of claim 4 wherein the biological sample is from a eukaryotic cell.

7. The method of claim 1 wherein the target RNA molecule comprises a label.

8. The method of claim 1 further comprising detecting the presence or absence of cleavage of the target RNA molecule.

9. The method of claim 1 further comprising resolving the sample after the incubation under conditions suitable to separate from the target RNA molecule the at least one cleaved RNA molecule comprising an adenosine at the 3′ terminal end.

10. The method of claim 9 wherein the conditions comprise denaturing polyacrylamide gel electrophoresis.

11. The method of claim 1 further comprising isolating the at least one cleaved RNA molecule comprising an adenosine at the 3′ terminal end.

12. A method comprising:
incubating a genetically modified cell, wherein the cell comprises an exogenous polynucleotide comprising a nucleotide sequence encoding a protein having A-specific RNAse activity, wherein the amino acid sequence of the protein and the amino acid sequence of SEQ ID NO:2 have at least 85% identity, and wherein the cell is incubated under conditions suitable for expression of the protein.

13. The method of claim 12 further comprising isolating the protein.

14. The method of claim 12 wherein the genetically modified cell is a bacterium or an archaeon.

15. The method of claim 14 wherein the genetically modified cell is a member of the genus Pyrococcus.

16. The method of claim 15 wherein the genetically modified cell is P. furiosus.

17. The method of claim 14 wherein the genetically modified cell is E. coli.

18. A genetically modified microbe comprising an exogenous protein, wherein the exogenous protein comprises an amino acid sequence, wherein the amino acid sequence and the amino acid sequence of SEQ ID NO:2 have at least 85% identity.

19. The genetically modified microbe of claim 18 wherein the exogenous protein comprises a heterologous amino acid sequence.

20. The genetically modified microbe of claim 9 wherein the heterologous amino acid sequence comprises a tag.
